(19) United States
(12) Patent Application Publication
(10) Pub. No.: US 2023/0301727 A1
(43) Pub. Date: Sep. 28, 2023

(54) DIGITAL GUIDANCE AND TRAINING PLATFORM FOR MICROSURGERY OF THE RETINA AND VITREOUS

(71) Applicant: Microsurgical Guidance Solutions, LLC, Chicago, IL (US)

(72) Inventors: Yannek I. Leiderman, River Forest, IL (US); Rogerio Garcia Nespolo, Warrenville, IL (US)

(21) Appl. No.: 18/328,914

(22) Filed: Jun. 5, 2023

Related U.S. Application Data
(63) Continuation-in-part of application No. 17/735,079, filed on May 2, 2022.
(60) Provisional application No. 63/183,424, filed on May 3, 2021.
(60) Provisional application No. 63/349,069, filed on Jun. 4, 2022.

(51) Int. Cl. A61B 34/20; A61F 9/008; A61B 34/00; G16H 40/63; G06V 10/25; G06V 20/50; G06T 11/60; G06T 7/20; G06V 20/70

(52) U.S. Cl. CPC A61B 34/20; A61F 9/008; A61B 34/76; G16H 40/63; G06V 10/25; G06V 20/50; G06T 11/60; G06T 7/20; G06V 20/70; A61B 2034/2065; A61B 2090/365

(57) ABSTRACT

An image-guided tool and method for ophthalmic surgical procedures is provided. The AI model develops operating image features based on the surgical instruments used in the region of interest and the phase of the surgical procedure being performed. Augmented visual images are then constructed that include the real-time visual image and the image features with additional features determined by the system.

### CROSS-REFERENCE TO RELATED APPLICATIONS

This application is a continuation-in-part of U.S. application Ser. No. 17/735,079, filed May 2, 2022, which claims priority to U.S. Provisional Patent Application No. 63/183,424, filed May 3, 2021, the disclosure of each of which is incorporated by reference in its entirety. This application also claims priority to U.S. Provisional Patent Application No. 63/349,069, filed Jun. 4, 2022, the disclosure of which is incorporated by reference in its entirety.

### BACKGROUND

Cataract extraction with lens implantation and vitrectomy procedures are among the most frequently performed ophthalmic surgeries in the United States and abroad. Although these procedures are generally considered safe and effective, surgical complications remain a cause of postoperative visual loss, including but not limited to retinal detachment, macular edema, intraocular bleeding, glaucoma, and permanent loss of vision.

All surgical procedures include inherent risk to the patient, the mitigation of which is an area of constant research and development. One advance in the field of medical practice has been to move to minimally invasive surgeries that do not require large incisions, and generally result in faster recovery, less pain, and less risk of complications. Many minimally invasive surgeries involve the insertion of one or more surgical instruments through one or more small incisions. Such surgeries generally rely on cameras, microscopes, or other imaging techniques (X-ray, ultrasound, etc.) in order for a surgeon performing the surgery to visualize the surgical field. However, one difficulty encountered during such procedures is that it can be difficult to interpret with accuracy visualization of the surgical field provided by medical imaging modalities to the surgeon.

In intraocular (i.e., within the eye) surgery, surgical optical visualization systems are used from outside of the eye to view the surgical field. Such visualization systems can include surgical microscopes (SM) or an optical coherence tomography (OCT) imaging system. OCT is an imaging technique that uses reflections from within imaged tissue to provide cross-sectional images.

### BRIEF DESCRIPTION OF THE DRAWINGS

FIG. 1 illustrates a block diagram of an example implementation of an image-guided surgical system (IGSS) according to this disclosure;

FIG. 2 illustrates an example schematic for a region-based convolutional neural network (R-CNN) according to this disclosure;

FIG. 3 illustrates a feedback loop formed by the components of the IGSS according to this disclosure;

FIG. 4 illustrates a method for developing and displaying image-guided tools for cataract and/or vitreoretinal surgical procedures according to this disclosure;

FIG. 5 illustrates an example display of an augmented image displayed during the capsulorhexis phase of a cataract surgical procedure according to this disclosure;

FIG. 6 illustrates an example display of an augmented image displayed during the phacoemulsification phase of a cataract surgical procedure according to this disclosure;

FIG. 7 illustrates an example display of an augmented image displayed during the cortex removal phase of a cataract surgical procedure according to this disclosure; and

FIG. 8 illustrates an example display of an augmented image displayed when no surgical instrument is inserted into the pupil during a cataract surgical procedure according to this disclosure.

FIGS. 9A and 9B show an example of image enhancement according to this disclosure.

FIG. 10 shows an example of concurrent tool and tissue tracking according to this disclosure.

FIG. 11 shows an example of tool and tissue tracking with automated laser control according to this disclosure.

FIG. 12 shows an example of concurrent tool and tissue tracking in which targeted scatter laser delivery is achieved via AI-guided laser delivery according to this disclosure.

FIG. 13 shows another example of concurrent tool and tissue tracking with collision avoidance according to this disclosure.

FIG. 14 shows an example of a system using a surgical procedure template according to this disclosure

### DETAILED DESCRIPTION

The figures, discussed below, and the various embodiments used to describe the principles of the present invention in this patent document are by way of illustration only and should not be construed in any way to limit the scope of the invention. Those skilled in the art will understand that the principles of the invention may be implemented in any type of suitably arranged device or system.

As used herein the term “real-time” refers to the acquisition, processing, and output of images and data that can be used to inform surgical tactics and/or modulate instruments and/or surgical devices during a surgical procedure, i.e., with sufficiently short delay that the output is usable as the surgery is performed, as disclosed in further detail herein.

Various systems and processes disclosed herein may be described with respect to manipulation of images, typically in real-time, for example to provide various augmentation of the images to a human operator. It will be understood that the same processes may be performed on a series of images, such as in a real-time video, to provide similar augmentation to a video stream. That is, manipulation and augmentation of “images” as disclosed herein also refers to such operations performed on a video, by performing those operations sequentially on a series of frames in the video.

Unfortunately, conventional OCT systems often lack the desired degree of precision regarding the precise location of the surgical instruments and tools and are adversely affected by imaging artifacts, such as shadows induced by the material properties of the surgical instruments. Additionally, delays in the visual output, due to computational complexities, may interfere with visualization of the surgical field in real-time. Visual images from a surgical microscope (SM) can be difficult for a surgeon to interpret with accuracy in real time due to the relationship between the surgical instruments and the anatomical structures and tissues in proximity to the surgical instruments particularly when the surgical field is exceedingly small, such as when operating on the eye.

The present disclosure provides image-guided tools and methods that use artificial intelligence (AI) models to process visual images to provide visual and other feedback to the surgeon for the guidance and positioning of the surgical instruments as well as to provide warning of eye movement and information on tissue segmentation.

During surgical procedures, unintended interactions between surgical instruments and tissues within the surgical field may have unintended consequences, some of which may be permanent. Ophthalmic microsurgery, for example, entails the use of mechanical and motorized instruments to manipulate delicate intraocular tissues. Great care must be afforded to tissue-instrument interactions, as damage to delicate intraocular structures such as the neurosensory retina, optic nerve, lens capsule, iris, and corneal endothelium can result in significant visual morbidity. The surgical guidance system described herein provides a feedback loop whereby the location of a surgical instrument in relation to delicate tissues (e.g., ocular tissues) and the effect of instrument-tissue interactions can be used to guide surgical maneuvers.

The present disclosure teaches embodiments capable of autonomously identifying the various steps and phases of phacoemulsification cataract surgery in real-time. The present disclosure uses an artificial intelligence (AI) model using a deep-learning neural network, such as for example, a region-based convolutional neural network (R-CNN), a segmentation network (SN), or the like to augment visual images from an operating room imaging system, such as, for example, a surgical microscope (SM) or an optical coherence tomography (OCT) imaging system. The AI model may intraoperatively identify the location and size of the pupil for tracking and segmentation, the surgical instruments used in the procedure that have entered the anterior chamber, capsular bag, vitreous body and posterior segment, and other physical components of the eye and/or instruments being used, as well as the surgical phase being performed and other details about the procedure itself. The AI model may provide augmented visual images to the surgeon in real time that show or identify the surgical instruments' location in the intraocular compartment, the phase of the surgical procedure, a suggested idealized tool path or other course of action to be performed by the surgeon using a tracked instrument, such as a path or location of an incision, target tissue or fragment to be cut or removed, site for application of laser energy, or the like, or other functional recommendation, and other information and features that may aid the surgeon during the surgical procedure. A system as disclosed herein also may display a surgical instrument's location, surgical phase, and/or other informational features in conjunction with or over visual images from the imaging system, preferably in real time.

The augmented images or overlayed images may be provided to the surgeon via a display device for the surgeon's information and use, for example, to the oculars of an SM, to a display monitor, or an augmented reality headset.

The present disclosure may use an image-guided surgical system (IGSS) to facilitate the delivery of real-time actionable image data and feedback to the surgeon during a surgical procedure. More specifically, the IGSS may generate and deliver computer-augmented images and/or other feedback to the surgeon allowing the surgeon to reliably recognize the location of surgical instruments and tissue boundaries in the surgical field and understand the relationship between the tissue boundaries and the surgical instrument.

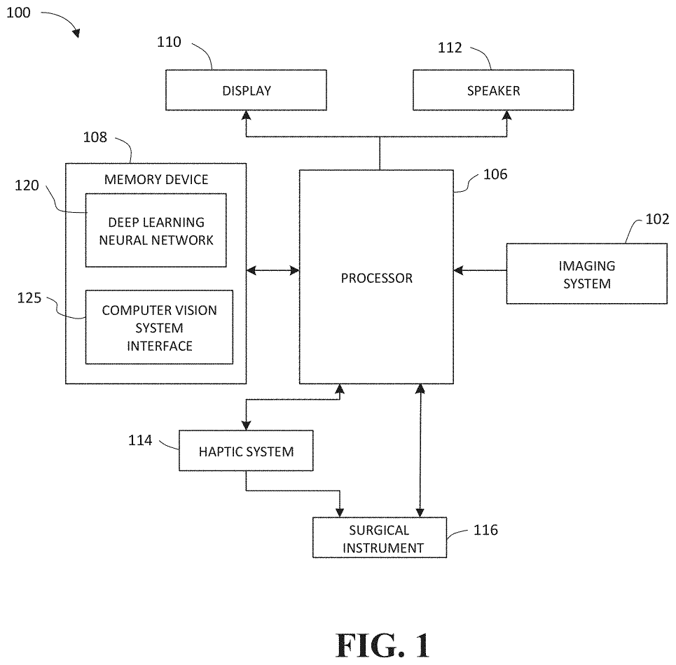

FIG. 1 is a block diagram illustrating an example IGSS used in the implementation of the disclosed embodiment. Generally, an IGSS 100 as disclosed herein includes an imaging system 102, one or more feedback devices 110,112 and 114, a computer processor 106, and a memory device 108. The feedback devices may include a display device 110, an audio speaker 112 or other noise-generating device, such as a piezoelectric buzzer and/or a haptic system 114, or the like. An IGSS 100 as disclosed herein may include the display device 110, though the display device 110 may provide more or less feedback to the surgeon, and may provide that feedback in a variety of forms, as described herein. For example, the IGSS 100 may display, via the display device 110, an image of the surgical field. The image of the surgical field may be augmented in one or more ways to depict, intermittently or continuously, within the surgical field, surgical instrument placement, selected features for the surgical phase, and/or surgical templates indicating, for example, a recommended tool path or incision location or course that guides the surgeon and classification of the surgical phase being performed, or the like. The augmentation may be provided in real-time. The IGSS 100 display device 110 may also display quantitative or qualitative information to the surgeon such as for example, movement or acceleration of surgical instruments and tissues, fluidic parameters such as turbulence, tissue mobility, and chamber instability, warnings regarding potentially unsafe conditions, such as deviation of a surgical instrument out of the field of view of the surgeon or imaging system, imminent collision of a surgical instrument with an intraocular structure or other tissue, likelihood of damage to or removal of tissues or structures intended to be preserved intact, or conditions of turbulent flow associated with surgical complications.

Even though the IGSS 100 is described using separate component elements in this disclosure, it will be well understood by those skilled in the art that in some arrangements certain elements of the IGSS 100 may be omitted and/or combined to provide the same functionality described herein. For example, the imaging system 102 may be integrated with the computer processor 106 and the display device 110. Alternately, the display device 110 may be a part of a stand-alone imaging system, not an integral part of the IGSS 100, that may be interfaced and used with an existing imaging system 102. The display device 110 may also include one or more display devices 110, audio speakers 112, haptic 114 or other feedback device for receiving augmented images and other feedback developed by the IGSS 100.

As was explained earlier, the present disclosure may use an AI model that utilizes a deep-learning neural network, such as a region-based convolutional neural network (R-CNN), a convolutional neural network (CNN), a segmentation network (SN), or the like, to augment visual images from the imaging system 102. For example, an AI model may be implemented in a deep learning neural network (NN) module 120 stored in memory device 108. The memory device 108 also may store a processor operating system that is executed by the processor 106, as well as a computer vison system interface 125 for constructing augmented images. When executing the stored processor operating system, the processor 106 is arranged to obtain the visual images of the surgical field from imaging system 102 and output augmented image data, using data provided by the NN 120. The augmented image data is converted to augmented images by the computer vision interface 125 and fed back to the surgeon on the display device 110. The processor 106, may also provide other forms of feedback to the surgeon such as audio alerts or warnings to the speaker 112, or vibrations or rumbles generated by the haptic system 114 to a surface of the imaging system 102 or to the surgical instrument 116. The audio warnings and vibrations alerting the surgeon of the surgical instrument 116 associated with a potential for suboptimal execution or complications such as for example, unintended deviation into a particular location or plane during the surgical procedure.

For example, the NN module 120 may provide object detection using a selected search for regions based on the following three processes:
1. Find regions in the image that might contain an object. These regions are called region proposals.
2. Extract convoluted neural network features from the region proposals.
3. Classify the objects using the extracted features.

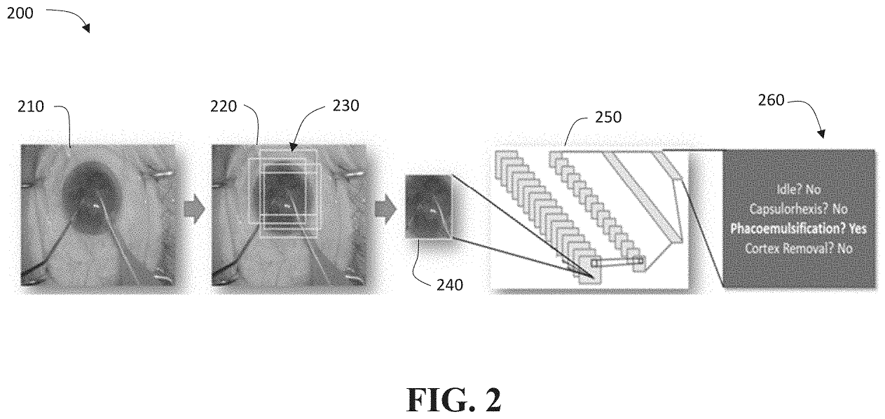

FIG. 2 illustrates a region-based convolutional neural network (R-CNN) algorithm 200 that can be used to develop augmented visual images. First an input image 210 is input to the R-CNN algorithm 200 from the imaging system 102. The R-CNN algorithm then generates region proposals 220 using an edge box algorithm 230. The R-CNN algorithm can produce at least 2000 region proposals. The individual region proposals 240 are fed into a convolutional neural network (CNN) 250 that acts as a feature extractor where the output dense layer consists of the features extracted from the input image 210. The extracted features identify the presence of the object within the selected region proposal generating the output 260 of the surgical phase being performed.

In another example, the NN module 120 may provide object detection using a semantic segmentation network. Semantic segmentation as used herein refers to a process of linking each pixel in a particular image to a class label. The class labels, for example within a surgical field, can be anatomical structures, tissue boundaries, instruments or instrument edges or boundaries, or the like. Through deep learning, the AI model can identify data sets of label images sampled from a training set of ophthalmic surgical procedures. Additionally, semantic segmentation of instruments enables creating an accurate profile of surgical instruments and usage across the surgical procedure. Such class label data sets, along with data for instrument trajectories, can serve as the basis for intraoperative image guidance, as well as image post-processing.

As will be described below, the imaging system 102 may include various devices that allow for suitable image capture, such as a two dimensions high-definition SM, a digital stereo microscope (DSM), or the like. The DSM is a surgical microscope that relies on at least two cameras that are offset. The two offset images are simultaneously displayed on a display device capable of three-dimensional display which confers stereo viewing to the user. In other embodiments, the IGSS 100 may include other surgical imaging systems such as an intraoperative optical coherence tomography (iOCT) system. In some cases, multiple types of imaging systems may be used, such as arrangements that include both an iOCT system and a SM or DSM system.

The display device 110 may take the form of one or more viewfinders such as the oculars of an SM or DSM, a high-definition display monitor, or a head-mounted display (such as those used for augmented reality and/or virtual reality systems), and the like.

The processor 106 typically is bi-directionally communicatively coupled to memory device 108, such that the processor 106 may be programmed to execute the software stored in the memory device 108 and, in particular, the visual images input to the IGSS 100 from imaging system 102.

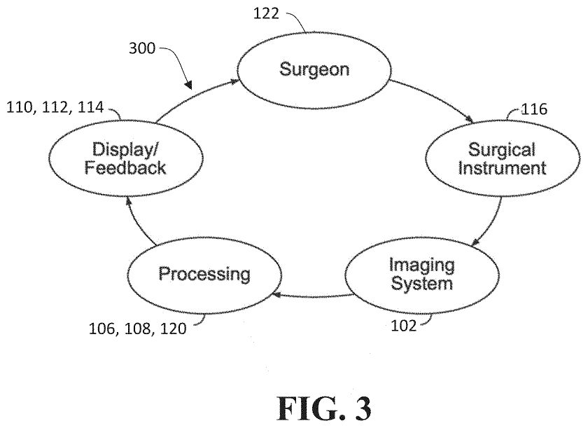

Together the elements of the IGSS 100 shown and described in FIG. 1 form a feedback loop 300, illustrated in block diagram by FIG. 3. In the feedback loop 300, a surgeon 122 provides input in the form of direct manipulation or robotic control to the surgical instrument 116. The surgical instrument 116 appears in the imaging system 102, which provides data to the processing system that comprises the processor 106, the memory device 108 and the NN module 120. The processing system post-process visual images from the imaging system 102 to output augmented images to the display device 110 and haptic feedback to haptic system 114, audio feedback to speakers 112 and/or to control features of certain surgical instruments used during the surgical procedure. The surgeon 122, based on the visual images or other feedback, may modify his or her actions accordingly or the processing system automatically may adjust certain features of a surgical instrument 116.

For example, based on the post processed visual images of the surgical instruments and tissue elements in the surgical field, various operational features of the surgical instrumentation 116 may be automatically adjusted by an automated or semi-automated process as disclosed herein. For example, the IGSS 100 may adjust the power to an ultrasonic phacoemulsification probe during emulsification of the lens nucleus during cataract surgery. The power driving the ultrasonics may be reduced, modulated, or shut-off in the event that suboptimal or high-risk conditions occur, or if the surgical instrument 116 exhibits unintended deviation into a particular location or plane, automatically, semi-automatically, and/or responsive to user input. As another example, the fluidics controller used in a cataract surgical system, used with phacoemulsification probes or irrigation-aspiration probes, and used to aspirate emulsified lens particles, lens material, or intraocular fluids may be automatically modulated by the feedback system to alter the vacuum generated by an associated vacuum pump based on detected changes in the behavior of tissues, surgical instruments, or other parameters of the surgical procedure. For example, the vacuum produced by the pump may be increased when the aspiration instrument is in the center of the surgical field removing hardened emulsified lens particles or decreased as it enters the softer outer lens cortex, in order to optimize instrument function.

As another example, embodiments disclosed herein may allow for adjusting the vacuum, flow, cutting rate, or duty cycle of a vitrectomy probe during pars plana vitrectomy. The parameters of the vitrectomy probe may be modulated or shut-off in the event that suboptimal or high-risk conditions occur, if the surgical instrument exhibits unintended deviation into a particular location or plane, or the like. Additionally, the fluidics controller used in a vitrectomy surgical system, the vitrectomy cutter, aspirating instruments, or other powered microsurgical tools used for surgery of the vitreous, retina, crystalline lens, or intraocular fluids may be automatically or semi-automatically modulated by the feedback system to alter the flow generated by an associated vacuum or flow pump based on detected changes in the behavior of tissues, surgical instruments, or other parameters of the surgical procedure. For example, the vacuum or flow produced by the pump may be increased when the aspiration instrument is in the center of the surgical field removing mobile core vitreous, or decreased as it approaches mobile retina in retinal detachment, peripheral retina, mobile lens capsule, or other tissues or structures to be excluded from excision or interaction with the surgical instruments.

In various embodiments of the IGSS 100 illustrated in FIGS. 1 and 3, the display device 110 may also include a depiction, in real-time, within the surgical field, of a surgical instrument 116 wielded by the surgeon, and may identify on the display 110 a tip, working end or other salient feature of the surgical instrument 116.

In another embodiment, the feedback from the processing subsystem may also be directly applied (not shown) to the surgical instrument 116 simultaneously as the augmented visual image is displayed to the surgeon 122. This would be useful in situations where the processing system detects a situation wherein eye structures, tissues, or spaces would be violated due to for example, shifting of tissue or patient movement. In such a scenario, haptic feedback such as for example, vibration or rumble could be sent to the surgical instrument 116 held in the surgeon's hand, providing tactile feedback as a warning to the surgeon. In robotic manipulated surgical instruments, the surgical instrument 116 can be automatically retracted from the area of concern by the motorized instrument manipulators or prevented from ingress into a particular zone of risk or prohibited location or plane. Alternately or in addition, in a haptic-mediated surgical system, resistance to movement of the surgical instrument could be induced by the haptic system 114 to prevent movement of the surgical instrument 116 into a particular zone of risk or prohibited location or plane.

As described above, the imaging systems 102 are located and used outside of the eye or body to view the surgical field. In some embodiments, particularly those configured to be employed during ocular surgeries such as cataract surgery, cornea surgery, glaucoma surgery, surgery of the retina and vitreous inclusive of laser procedures during incisional surgery, and the like, the imaging systems may include an SM system, a DSM system an iOCT system, or combination of such imaging systems. The imaging system 102, when configured to be employed during ophthalmic surgeries, may be configured and operative to identify any of a variety of ocular tissues, including optic nerve, retinal vessels, macula, macular hole, retinal holes, retinal tears, retinal detachment, scleral depression mound, the ora serrata, vitreous hemorrhage, retinal laser spots (both fresh and scarred lesions), retinal laser aiming beam, lens tissue, cracking defects in the lens fragments, corneal tissue, the corneal limbus, iris tissue, the pupil, the anterior chamber, the capsulorrhexis margins, the hydrodissection light reflex, position and centration of the intraocular lens implant, pharmacologic agents used for particulate visualization of the vitreous, pharmacologic agents used to stain or identify the internal limiting membrane and epiretinal proliferative tissue (including epiretinal membrane and proliferative vitreoretinopathy), intraretinal hemorrhage, retinal drusen, geographic atrophy lesions, lattice degeneration, the pars plana, choroidal detachment, lens tissue, the lens capsular bag, the cornea and individual corneal lamellae, the iris, the pupillary margins, the trabecular meshwork, Schlemm's canal, the fundus red reflex, position and centration of the intraocular lens implant, and surgical instruments including but not limited to the vitrectomy probe, fiberoptic endoilluminator, extrusion cannula, intraocular forceps and scissors, subretinal injection needle, vascular cannulation needles and related devices, endolaser probe, retinal picks and scrapers, intraocular OCT probe, endoscopes, phacoemulsification probe, irrigation and aspiration devices, lens chopping instruments and related cannulas, needles, and devices for injection of pharmacologics and injectable drugs or devices during ocular surgery, or combinations thereof.

In some embodiments, in parallel focus assessment may be performed in which feedback on the optimal image focus may be provided to the surgeon, or directly to the SM, DM, or other imaging device in order to assess or optimize image focus. For example, out-of-focus objects may be detected using computer vision and/or neural network algorithms of the network-detected and segmented surgical instruments and tissues, providing feedback to the surgeon to facilitate optimal visualization, or directly to visualization instrumentation.

In the presently described embodiment, visual images in the form of digital image data from the imaging system 102 is input into the NN module 120 of the AI model. The NN module 120 may analyze the digital image data to determine the tissue boundaries and/or layers, so that the data output by the AI model indicates the tissue boundaries/layers that may be added to the raw image data for display to the surgeon. The displayed tissue boundaries/layers assisting the surgeon in avoiding contact between the surgical instrument 116 and sensitive tissues. Additionally, the AI model may also provide instrument guidance for spatial orientation and/or optimizing instrument parameters related to function such as aspiration and associated vacuum/flow rates, ultrasound parameters, etc.

The AI model may automatically segment the image data using a deep learning approach using the algorithm of the NN module 120. The AI model receives as an input the digital image data obtained from the imaging system 102 and provides a segmentation probability map of the location of the tissue in question (e.g., the retina, the lens, etc.). The segmentation probability map may also provide utility measurement, such as, for example, the relative area change and/or volume change of the tissue of the retina or the lens between different images. The utility measurement of the area of change/or volume of change may be used by the surgeon to estimate how much the tissue's area is changing, therefore providing information about the amount of stress the tissue is undergoing at a particular instant in the procedure. The relative change in height of the tissue between images provides a similar, but different, type of stress indication. The position and/or motion of the tissue relative to adjacent ocular tissues, identify occlusion of an instrument by a tissue. Using the algorithms described herein, the segmentation may be achieved at a frame rate of up to or in excess of 60 frames-per-second, thereby allowing the presentation to the surgeon of real-time segmented images as augmented visual images.

The NN module 120 algorithms of this embodiment use datasets, with training performed in a supervised or self-supervised manner, which include both the source visual image and the segmented images. The trained AI model implementing the NN module 120 uses digital image data from the imaging system, labelled by experts, as a training set, in the case of supervised learning.

In still another embodiment, the AI model may also be used to pre-process the input visual images received from the imaging system 102. The Pre-processing of the input images improves image resolution for the surgeon for use in real time, as a form of image enhancement to allow the surgeon to appreciate details of the image that may otherwise be obscured or not apparent in un-processed imaging.

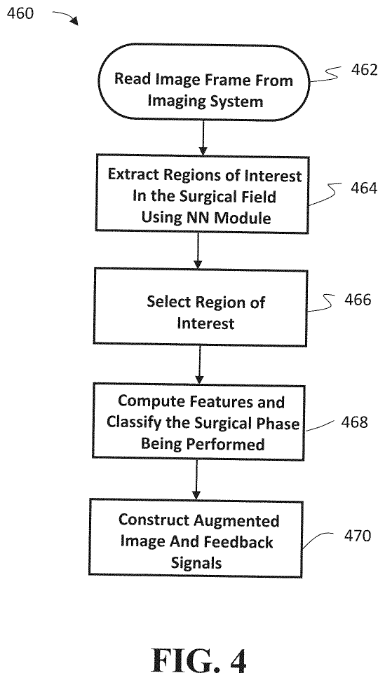

FIG. 4 illustrates a flow chart depicting a method 460 that implements an example process as disclosed herein for cataract and/or vitreoretinal surgical procedures employing an image-guided tool of the present disclosure. In step 462, the processor 106 receives digital image data representing visual images captured in real-time from the imaging system 102. The digital image data along with the AI trained data from the NN module 120 is input to the processor 106 in step 464. The processor 106 identifies and outputs data identifying region proposals for the pupil's location and area as described earlier in the discussion for R-CNN.

In step 466, the processor 106 using a calculated R-CNN classification for the components and structures of the eye in the field, selects a region of interest based on an optimal operable location and area. The processor 106 using the AI trained data from NN module 120 computes in step 468, selected features for the surgical phase and identifies the classification of the phase of the surgical procedure being performed. The features and classification may be based on the type of surgical instruments located within the image captured by the imaging system 102.

In step 470 augmented visual images are constructed by the computer vision systems interface 125. The augmented visual images are output to the surgeon's SM eyepiece or the display device 110. Additionally, feedback signals may also be output including haptic and/or audible signals applied to the haptic system 114 or speaker 112. The method described by steps 462-470, are made for each image frame captured from the imaging system 102, always acquiring the last available frame from the imaging system 102 at a minimum video streaming rate of 60 frames per second, or other frame rates that mat be suitable.

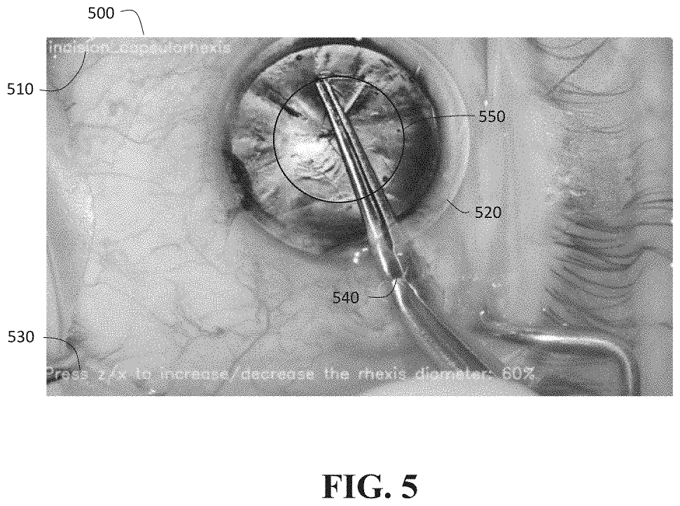

The feedback returned by the imaging system may include guidance for the optimal creation of a capsulorrhexis, including size, location, centration, and symmetry of the rhexis as is seen in FIG. 5. Capsulorrhexis parameters and guidance recommendations may be altered in real time based upon real-time image-based feedback on the evolving capsulorrhexis as executed during the surgery. The augmented visual image 500 provides an identification of the phase of the surgical procedure 510, based on the type of surgical instrument 540 used in the procedure. Image 500 displays a rhexis template 550 and guidance instructions 530 to guide and instruct the surgeon for adjustment of the rhexis diameter as features for this surgical phase. Further features include visual feedback of the pupil boundary 520 where local contrast enhancement may be applied. FIG. 5 illustrates the surgical instrument 540 penetrating the outer tissue of the eye, either the sclera or the cornea.

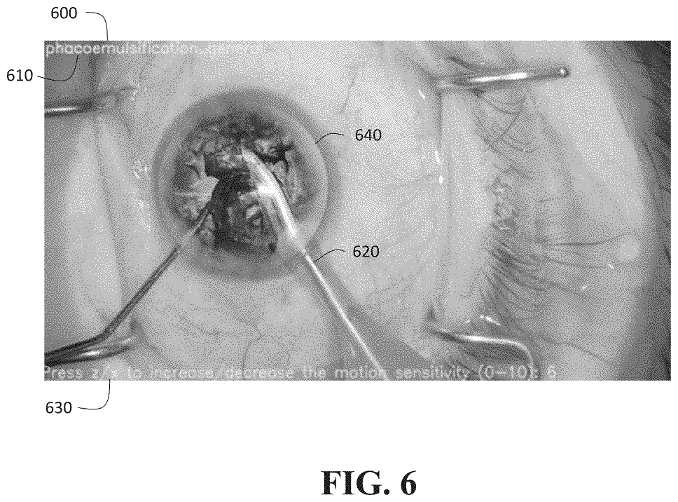

FIG. 6 displays the augmented visual image 600 for surgical guidance during disassembly and removal of the lens nucleus via phacoemulsification. Identification of the procedure is identified at 610 based on the surgical instrument 620 used in the procedure in this surgical phase, excessive eye movement warning, computation of amount of remaining lens fragments via tissue segmentation, and estimation of turbulent flow conditions via tracking of the motion of lens fragments may be visually indicated to the surgeon by displaying a visual cue 640 where local contrast enhancement may be applied. Visual cue 640 indicates when turbulence or any brusque movement of the surgical instrument 620 is identified. The feedback thresholds for the guidance parameters 630 may be modulated by the surgeon and are provided as a visual feature, during the relevant surgical phase.

The tracking of turbulent flow during this surgical phase uses visual feedback from the NN module 120 or from computer vision techniques that estimate turbulent flow, the movement and tracking of the surgical instruments and lens fragments. Biomarkers associated with surgical risk may also be detected as features and information provided to the surgeon in this surgical phase. For example, rapid changes in pupillary size, reverse pupillary block, trampoline movements of the iris, spider sign of the lens capsule, and a change in the fundus red reflex may be identified and provided as feedback in real time to the surgeon. In addition, instrument positioning associated with surgical risk, such as decentration of the tip of the phacoemulsification needle outside of the central zone, duction movements of the globe away from primary gaze during surgery and patient movement relative to the surgical instruments may be identified and provided as feedback to the surgeon as either visual warning images, or haptic and audio alarms in real-time.

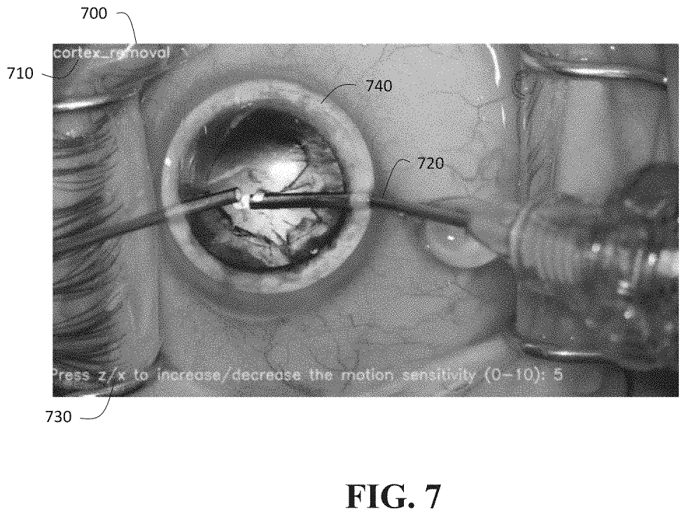

FIG. 7 displays the augmented image 700 for cortex removal. Based on the instrument 720 used, feedback information is presented to the surgeon including the procedure phase 710. Instrument 720 movement warnings and motion sensitivity thresholding 730 is also provided to aid in the removal of cortical fibers. The augmented image 700 can have contrast equalization applied in the form of a local image enhancement 740 where local contrast enhancement is applied. As is seen in FIG. 7, a visual cue 740 is applied within and around the area of the pupil.

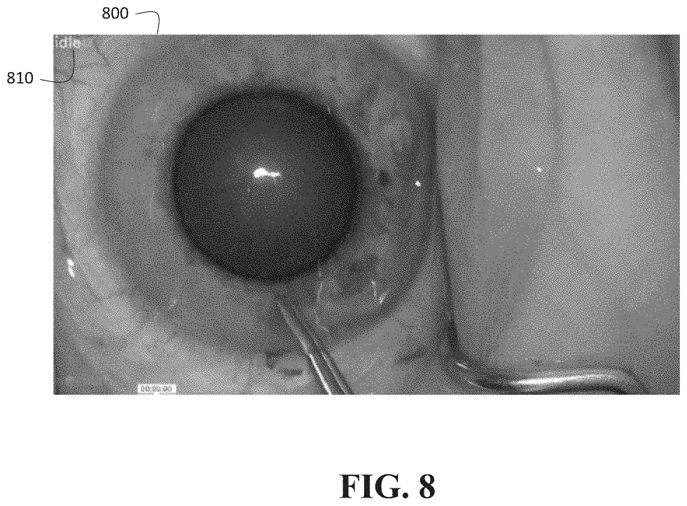

When no instrument is inserted into the pupil, the CNN recognizes the image 800 phase being performed as “idle” as shown at 810 of FIG. 8.

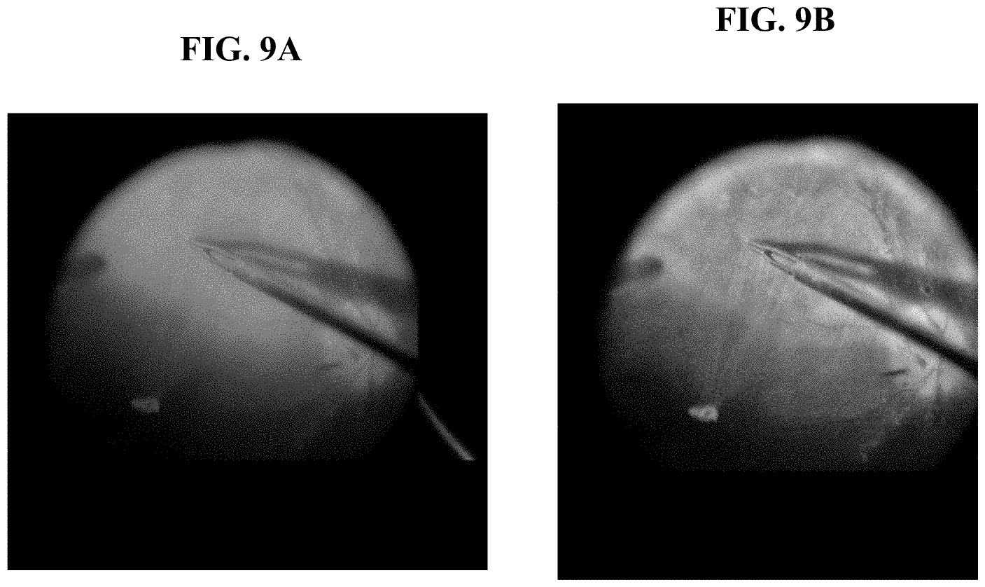

Embodiments disclosed herein also may provide general enhancement of video and other images provided to a surgeon during a procedure. FIGS. 9A and 9B show an example of enhancement applied to an image captured during a surgery as disclosed herein. FIG. 9A shows an unenhanced image captured from a DSM, in which some retinal features are indistinct in the surgeon's view. The image may be, for example, a still frame of a continuous video feed available for use by a system as disclosed herein during the performance of a surgical operation. FIG. 9B shows the same view after being processed using artificial intelligence-based image enhancement technology as disclosed herein. As an illustrative example, a surgical enhancement system and process as disclosed herein may use contrast-limited adaptive histogram equalization to generate an image as shown in FIG. 9B from the image shown in FIG. 9A. Other techniques and tools may be used. The enhancement improves the resolution and visualization of retinal features and tissues, thereby providing the surgeon with an improved view of the surgical field. The enhanced image may be injected into the surgeon's view in the SM, DSM, or other visualization system to facilitate visualization during surgery. As previously disclosed, the enhanced image may be provided in real time during performance of the surgery.

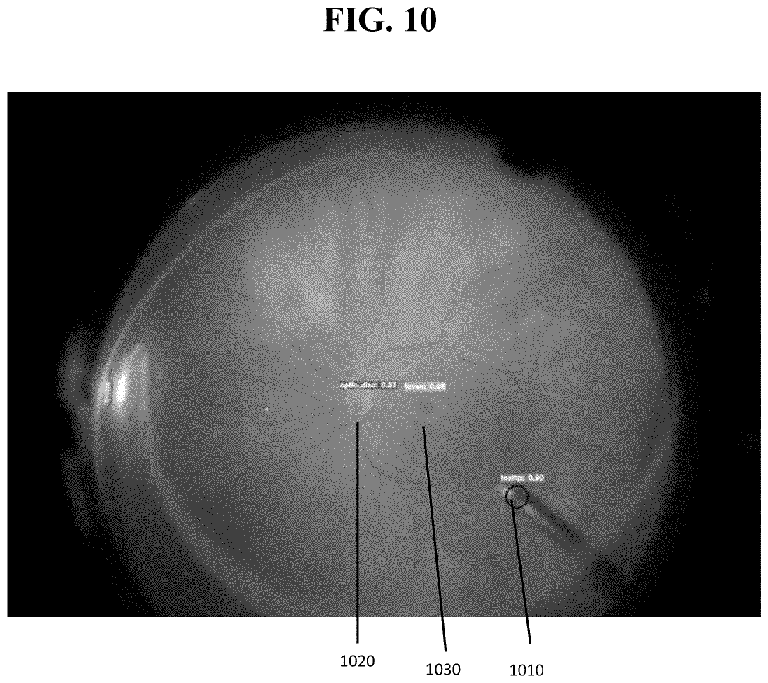

Embodiments disclosed herein provide devices, systems, and techniques that can concurrently identify and segment relevant anatomical structures and landmarks, biological tissues, and the like, as well as surgical devices and instruments, in real time during a procedure. FIG. 10 shows such an example of concurrent tool and tissue tracking as disclosed herein. In this example, AI-based automated image segmentation and recognition is being performed by an IGSS using techniques as previously disclosed. A surgical instrument, the vitrectomy probe 1010, is shown, as well as two tissue elements—the optic nerve 1020, and the macula 1030. The instrument 1010 and the tissue elements 1020, 1030 are identified and continuously tracked within the surgical frame in real time, Text labels and/or colored overlays may be applied to the tool and tissue elements as shown to identify them to the surgeon. As previously disclosed, various other information may be provided, such as where a warning may be displayed on the screen or via an audible alert if the tool 1010 is moved to an area where it may cause damage or an otherwise undesirable position.

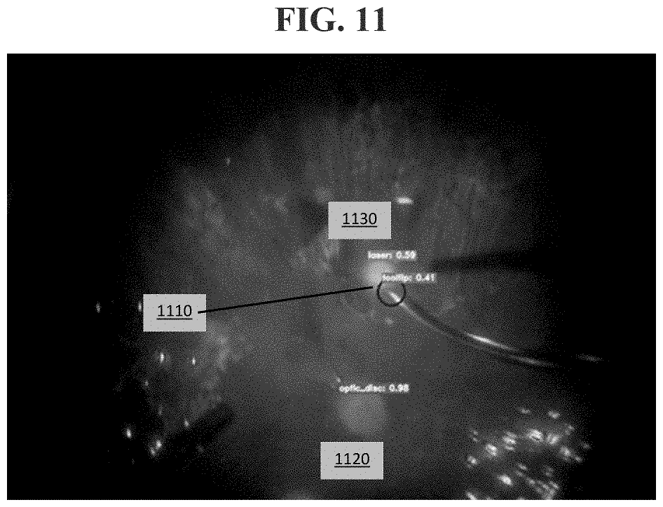

In some use cases, an AI-based IGSS system as disclosed herein may allow for automated control of laser treatment that is originally placed or initiated by a surgeon. FIG. 11 shows another example of tool and tissue tracking according to the present disclosure that uses automated laser control. As in previous examples, an AI-based automated image segmentation and recognition is used to identify surgical instruments, including an endolaser probe 1110 and, concurrently the optic nerve 1120, and laser spots 1130 as they are applied. Each identified component is segmented as previously disclosed, and the system may continuously track them within the surgical frame in real time. Identifying laser spots at the margin of the break as they are applied provides the foundation for semi-automated delivery of the retinal laser. For example, the surgeon may manually direct the endolaser probe and associated aiming beam at the margin of a retinal break. The system then automatically detects the margin of the retinal break where the laser is to be applied to seal the break, and delivers the laser in a discrete or continuous pattern. As laser reaction is successfully achieved (causing the white spots in the figure), the system automatically identifies the margin of the retinal break where the laser has been applied adequately. Feedback to the laser delivery system prevents subsequent delivery of additional laser to previously treated sites. Once the aiming beam traverses to the untreated retina at the margin of the break, the feedback causes in re-activation of the laser to achieve retinopexy. In this way, efficient, targeted laser delivery is achieved via aiming of the laser probe by the surgeon in combination with automatic laser-guided delivery by the AI-based system as disclosed.

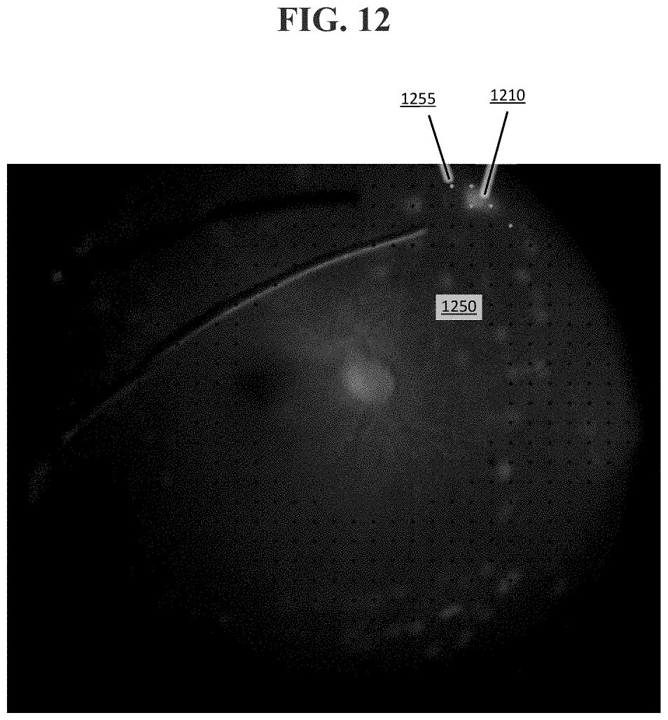

FIG. 12 shows another example of concurrent tool and tissue tracking, in which targeted scatter laser delivery is achieved via AI-guided laser delivery by the system after initial aiming of the laser probe by the surgeon. Segmentation, recognition, and tracking is performed as previously disclosed. In this example, an endolaser probe is recognized and tracked. Concurrently, laser spots (burn lesions) are identified as they are applied and may be continuously tracked within the surgical frame in real time.

Concurrently or sequentially, to facilitate panretinal photocoagulation, a customizable template 1250 for scatter laser may be generated, sparing the disc, arcade vessels, macula, and other critical anatomy (as shown by the black dots). As the surgeon sweeps the laser aiming beam across the target area, the network identifies areas of overlap between the aiming beam and the template 1250, and then delivers a laser spot at the overlap. The individual laser-induced retinal spots are recognized and registered by the AI system, as noted by the template points changing from black to green 1255. A feedback loop to the laser delivery system as previously disclosed herein allows for rapid, semi-automated panretinal laser photocoagulation. The laser is only activated when there is overlap with the laser aiming beam and template, allowing the surgeon to continuously sweep the laser aiming beam throughout the intended target area and still achieve the desired pattern and spacing of thermal-laser-induced spots. In this way, efficient, targeted scatter laser delivery is achieved via aiming of the laser probe by the surgeon combined with laser-guided delivery by the AI-network.

Various other templates may be used for other surgical procedures. Templates may provide overlays, distance and operational information, and other information specific to one or more procedures, or they may provide general information such as a distance measurement provided by a digital caliper as disclosed in further detail below. Templates may be stored in a template library of the AI-based system, for example in a computer-readable storage medium accessible by a processor of the system. A surgeon may select or provide an appropriate template for a particular surgical procedure.

Real-time tissue and tool tracking during surgery as disclosed herein allows for a number of surgical applications, including but not limited to collision avoidance, in which a warning or other signal is provided to the surgeon if a surgical instrument or tool comes into proximity or contact with a tissue or structure that has been identified as an exclusion zone. For example, the retinal surface may be defined as a zone of exclusion relative to the vitrectomy probe, fiberoptic endoilluminator, laser probe, forceps, scissors, picks, cannulas, needles, intraoperative OCT probe, or endoscopes, in order to prevent damage from contact with surgical instruments. Automated continuous tool and tissue tracking also has applications in surgical training and surgical data science. Quantitative assessment of the path and behavior of instruments and tissues during surgery confers the potential to apply analytics to the execution of surgery to assess performance, progress in surgical training, conduct predictive analytics on surgical risk/complications, and to provide insights into the behaviors and patterns of surgeons possessing varying degrees of ability. Identifying patterns and features associated with successful execution and outcomes, as well as features associated with risk or complications, may further serve to inform network performance and improve the capabilities of guidance systems.

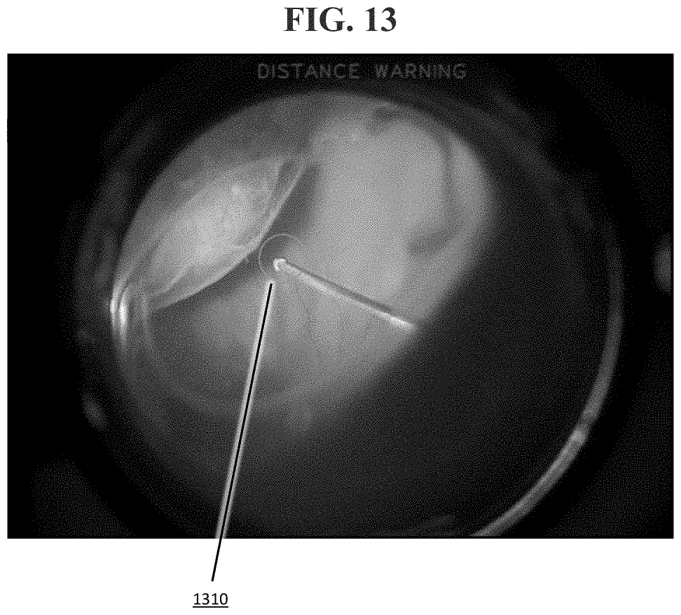

FIG. 13 shows another example of concurrent tool and tissue tracking as disclosed herein, used to provide such a feature. In this example, a vitrectomy probe 1310, and two tissue elements, the scleral depression mound and area of retinal detachment, are identified, segmented, and continuously tracked within the surgical frame in real time, as previously disclosed. When the vitrectomy 1310 probe comes into proximity to mobile detached retina, a proximity alert (‘Distance Warning’, as shown at the top of the figure) is provided to the surgeon, for example via a visual overlay, audible alert, haptic feedback, or the like. The threshold for engaging the proximity alert may be customized by the surgeon, and also may be determined during the surgery based on, for example, tissue and instrument parameters. For example, a mobile and bullous retina is more easily unintentionally aspirated by the vitrectomy probe, and high amplitude- or variable mobility of a detached retina may be assessed by the system as features that would modulate the threshold for proximity alert. Similarly, increasing vacuum or flow via the fluidics module would similarly result in modulation of the threshold for proximity warning.

As another example, activation of a proximity alert may result in feedback directly to the surgical device control systems, so that surgical parameters such as vacuum, flow, duty cycle, or on/off functionality of a guillotine cutter or similar device is modulated via direct feedback from the image-processing network or the AI-based system as a whole. For example, a feedback loop directly between the image-processing system and the surgical instrumentation system may modulate instrument performance. Such feedback may occur rapidly, in some cases equally quickly or more quickly than the limits of human reaction time, thereby potentially averting unintended consequences of tissue-instrument interactions.

As another example, instrument positioning associated with surgical risk, such as decentration of the tip of a surgical instrument outside of the visualized zone, proximal to tissues defined as exclusion zones, or patient movement relative to the surgical instruments may be identified and provided as feedback to the surgeon as visual warning images, haptic feedback, audio alarms, or the like, or combinations thereof; or the feedback may be provided directly to the surgical instrumentation in real-time as previously disclosed.

As another example, the spatial resolution of instrument detection may be enhanced via the incorporation of electromechanical sensors into the surgical instrument itself, including but not limited to tools such a vitrectomy probes, fiberoptic endoilluminators, extrusion cannulas, intraocular forceps and scissors, subretinal injection needles, vascular cannulation needles and related devices, endolaser probes, retinal picks and scrapers, intraocular OCT probes, endoscopes, phacoemulsification probes, irrigation and aspiration devices, lens chopping instruments, and related cannulas, needles, and devices for injection of pharmacologics and injectable drugs or devices during ocular surgery. The integration of spatial data from electromechanical sensing devices together with image-based positional data from an AI-based system as disclosed herein may provide for greater accuracy and precision of surgical tool- and tissue-tracking than is possible by a human operator alone, or by an AI-based system operating without such devices.

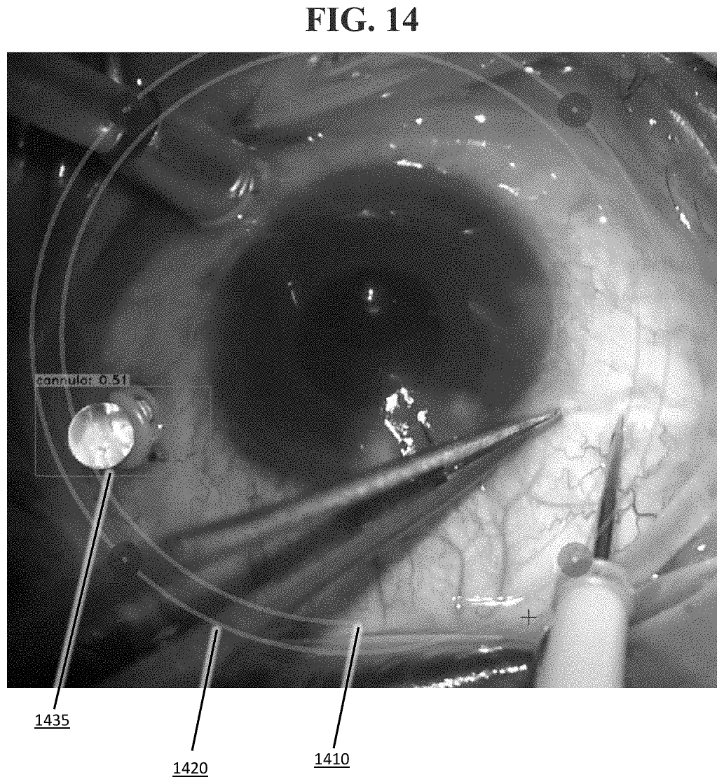

As previously disclosed with respect to FIG. 12, surgical templates may allow for improved placement, manipulation, and operation of surgical tools and operation of surgical procedures. FIG. 14 shows another example of surgical templates used in a system as disclosed herein. AI-based automated image segmentation and recognition may be performed as previously disclosed. In this example, the surgical limbus, a tissue element, is identified and continuously tracked within the surgical frame in real time as previously disclosed. Using a conventional approach, physical calipers would be used by the surgeon to measure the desired distance from the edge of the cornea to make suitable incisions, typically 3.5 mm or 4.0 mm in this example procedure, though any distance may be used in various other procedures and arrangements. In a system as disclosed herein, instead of requiring the use of a physical measuring device, a customizable digital “caliper” is generated by the network and injected into or overlayed onto the image view provided to the surgeon. For example, the system may generate two calipers 1410, 1420, showing the two conventional incision radii at 3.5 mm and 4.0 mm, respectively. In addition to freeing the surgeon's hands and attention from manipulating the physical calipers, this arrangement may be more accurate and repeatable than conventional techniques. It also allows for the incision(s) to be made at any point around the caliper rings, allowing the surgeon to position the tools in a desirable position, for example where it can be held most steady, where access to the surgical site is most convenient, safest, or allowing for the maximum degrees of freedom of surgical instrument handling, or the like. As previously disclosed, tools also may be identified and tracked, including via overlay labels or the like, such as the cannula 1435. Digital calipers as shown in this example may be used more generally for other procedures, to help identify target sites for surgical incisions and placement of microsurgical trocars.

The various features, improvements, and arrangements shown and disclosed herein may be used in any combination. For example, the various overlays, alerts, and other feedback mechanisms may be used in any desired combination for any procedure in which AI-based analysis and manipulation of images and video signals as described herein may be used.

As explained above, embodiments disclosed herein include methods of operating a surgical system. Such a method may include features such as receiving a series of visual images from an imaging system of a surgical field; extracting one or more regions of interest in the surgical field using information provided by an AI model based on the series of visual images; identifying a surgical tool in the region(s) of interest; identifying a tissue element in the region(s) of interest; tracking the relative placement of the surgical tool and the tissue element; and providing feedback to a human operator of the system based on the relative placement of the surgical tool and the tissue element. The images may be received and/or processed in real time.

Various forms of feedback may be provided. For example, the feedback may include an augmentation of the series of visual images such as a visual label identifying the surgical tool, a label identifying the tissue element, or both; a visible, audible, or combined proximity warning indicating that the surgical tool is too close to the tissue element; an indication that the surgical tool is misplaced; a template overlay on the series of images, for example to indicate one or more placements of the surgical tool to perform a surgical procedure, such as visual indication of regions for application of a laser treatment or a visual indication of one or more suitable places for an incision or surgical removal of a portion of tissue; a clarification of the focus of the image, such as to make the image more clear for the surgeon; or any combination thereof.

Embodiments also provide devices and systems for performing ophthalmic surgical procedures, which may include a surgical tool; a computer processor; a display device coupled to the computer processor; an imaging system coupled to the processor; and a memory device, coupled to the processor, storing instructions executable by the processor to operate an artificial intelligence (AI) model. The AI model may be configured to perform any of the calculations, analyses, and augmentations disclosed herein. For example, the model may receive a series of visual images of a surgical field from the imaging system and extract regions of interest in the surgical field using information provided by an artificial intelligence (AI) model based on the series of visual images. The AI model, the computer processor, or a combination thereof may further be configured to identify the surgical tool in the region(s) of interest; identify a tissue element in the region(s) of interest; track the relative placement of the surgical tool and the tissue element; and provide feedback to a human operator of the system based on the relative placement of the surgical tool and the tissue element. The surgical tool may be communicatively coupled to the processor and configured to operate automatically based upon a signal received from the processor.

The system also may include a template library storing surgical templates, each of which provides augmentation data for one or more surgical procedures. Templates may include, for example, one or more placements of the surgical tool to perform a surgical procedure; a visual indication of regions for application of a laser treatment; a visual indication of suitable placement for an incision or surgical removal of a portion of tissue; or any combination thereof.

The system may generate and provide an augmentation of the images provided to the surgeon, which may include, for example, an overlay on the series of images defined by a surgical template selected from the template library. The augmentation may include one or more of a visual label identifying the surgical tool, a label identifying the tissue element, or a combination thereof; a proximity warning indicating that the surgical tool is too close to the tissue element; and an indication that the surgical tool is misplaced.

The surgical tool may include a haptic feedback mechanism, for example to provide feedback on the procedure being performed based on the identification and tracking of the placement of the tool.

In some embodiments, various functions described in this patent document are implemented or supported by a computer program that is formed from computer readable program code and that is embodied in a computer readable medium. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

It may be advantageous to set forth definitions of certain words and phrases used throughout this patent document. The terms “application” and “program” refer to one or more computer programs, software components, sets of instructions, procedures, functions, objects, classes, instances, related data, or a portion thereof adapted for implementation in a suitable computer code (including source code, object code, or executable code). The term “communicate,” as well as derivatives thereof, encompasses both direct and indirect communication. The terms “include” and “comprise,” as well as derivatives thereof, mean inclusion without limitation. The term “or” is inclusive, meaning and/or. The phrase “associated with,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, have a relationship to or with, or the like. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C.

The description in the present application should not be read as implying that any particular element, step, or function is an essential or critical element that must be included in the claim scope. The scope of patented subject matter is defined only by the allowed claims. Moreover, none of the claims is intended to invoke 35 U.S.C. § 112(f) with respect to any of the appended claims or claim elements unless the exact words “means for” or “step for” are explicitly used in the particular claim, followed by a participle phrase identifying a function. Use of terms such as (but not limited to) “mechanism,” “module,” “device,” “unit,” “component,” “element,” “member,” “apparatus,” “machine,” “system,” “processor,” or “controller” within a claim is understood and intended to refer to structures known to those skilled in the relevant art, as further modified or enhanced by the features of the claims themselves, and is not intended to invoke 35 U.S.C. § 112(f).

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure, as defined by the following claims.

What is claimed is:

1. A method of operating a surgical system, the method comprising:
receiving a series of visual images from an imaging system of a surgical field;
extracting a plurality of regions of interest in the surgical field using information provided by an artificial intelligence (AI) model based on the series of visual images;
identifying a surgical tool in a first region of interest of the plurality of regions of interest;
identifying a tissue element in a second region of interest of the plurality of regions of interest;
tracking the relative placement of the surgical tool and the tissue element; and
providing feedback to a human operator of the system based on the relative placement of the surgical tool and the tissue element.

2. The method of claim 1, wherein the feedback comprises:
an augmentation of the series of visual images.

3. The method of claim 2, wherein the augmentation comprises a visual label identifying the surgical tool, a label identifying the tissue element, or a combination thereof.

4. The method of claim 2, wherein the augmentation comprises a proximity warning indicating that the surgical tool is too close to the tissue element.

5. The method of claim 2, wherein the augmentation comprises an indication that the surgical tool is misplaced.

6. The method of claim 2, wherein the augmentation comprises a template overlay on the series of images, the template indicating one or more placements of the surgical tool to perform a surgical procedure.

7. The method of claim 6, wherein the template comprises a visual indication of regions for application of a laser treatment.

8. The method of claim 6, wherein the template comprises a visual indication of one or more suitable places for an incision or surgical removal of a portion of tissue.

9. The method of claim 2, wherein the augmentation comprises a clarification of the focus of the image.

10. The method of claim 1, wherein the series of visual images is received and processed in real time.

11. A system for performing an ophthalmic surgical procedure, the system comprising:
a surgical tool;
a computer processor;
a display device coupled to the computer processor;
an imaging system coupled to the processor;
a memory device, coupled to the processor, storing instructions executable by the processor to operate an artificial intelligence (AI) model configured to receive a series of visual images of a surgical field from the imaging system;
wherein the instructions further cause the computer processor, the AI model, or a combination thereof to:
extract a plurality of regions of interest in the surgical field using information provided by an artificial intelligence (AI) model based on the series of visual images;
identify the surgical tool in a first region of interest of the plurality of regions of interest;
identify a tissue element in a second region of interest of the plurality of regions of interest;
track the relative placement of the surgical tool and the tissue element; and
provide feedback to a human operator of the system based on the relative placement of the surgical tool and the tissue element.

12. The system of claim 11, wherein the surgical tool is communicatively coupled to the processor and configured to operate automatically based upon a signal received from the processor.

13. The system of claim 11, further comprising a template library storing a plurality of surgical templates, each surgical template providing augmentation data for one or more surgical procedures.

14. The system of claim 13, wherein the augmentation comprises an overlay on the series of images defined by a surgical template selected from the plurality of surgical templates.

15. The system of claim 14, wherein the surgical template indicates one or more placements of the surgical tool to perform a surgical procedure.

16. The system of claim 14, wherein the template comprises a visual indication of regions for application of a laser treatment.

17. The system of claim 16, wherein the template comprises a visual indication of one or more suitable places for an incision or surgical removal of a portion of tissue.

18. The system of claim 11, the surgical tool further comprising a haptic feedback mechanism, wherein the surgical tool is configured to actuate the haptic feedback mechanism responsive to a signal from the processor.

19. The system of claim 11, wherein the augmentation comprises one or more items selected from a group consisting of: a visual label identifying the surgical tool, a label identifying the tissue element, or a combination thereof; a proximity warning indicating that the surgical tool is too close to the tissue element; and an indication that the surgical tool is misplaced.
